(19) United States
(12) Patent Application Publication
(10) Pub. No.: US 2021/0319849 A1
(43) Pub. Date: Oct. 14, 2021

(54) METHOD FOR ASSESSING GENOME ALIGNMENT BASIS

(71) Applicant: KONINKLIJKE PHILIPS N.V., ElNDHOVEN (NL)

(72) Inventor: Vartika Agrawal, Cambridge, MA (US)

(21) Appl. No.: 17/272,517

(22) PCT Filed: Aug. 21, 2019
(86) PCT No.: PCT/EP2019/072327

§ 371 Date: Mar. 1, 2021

Related U.S. Application Data
(60) Provisional application No. 62/723,676, filed on Aug. 28, 2018.

(51) Int. Cl. G16B 30/10; G16B 20/20; G16B 45/00

(52) U.S. Cl. CPC G16B 30/10; G16B 45/00; G16B 20/20

(57) ABSTRACT

A method (100) for analyzing a target genome, comprising: (i) aligning (120) sequencing data from the target genome to a reference genome; (ii) identifying (130) heterozygous locations, comprising an identification of allele variants and a frequency of each allele, the allele variants comprising both a reference allele variant and a non-reference allele variant; (iii) generating (140) an alternate reference genome, wherein the identified non-reference allele variant for the identified heterozygous locations replaces the reference allele variant in the reference genome; (iv) aligning (150) sequencing data to the alternate reference genome; (v) identifying (160) a frequency of the allele variants at each of the heterozygous locations; (vi) assessing (170) alignment bias at the identified heterozygous locations, comprising comparing the frequency of allele variants from the reference genome alignment to the frequency of allele variants from the alternate genome alignment; and (vii) generating (190) a report comprising the assessment of alignment bias.

### FIELD OF THE DISCLOSURE

The present disclosure is directed generally to methods and systems for assessing alignment bias in next-generation sequencing analysis.

### BACKGROUND

Many next-generation sequencing methodologies fragment DNA and generate sequencing reads which are then aligned to a reference genome. This identifies sites of variation in the analyzed genome relative to the reference genome. The human genome, for example, comprises approximately one million polymorphic locations where there is evidence of two of more alleles across different populations.

All downstream analysis of an alignment is contingent upon the accuracy of the alignment and the correct identification of variation within the sequenced genome. However, since a reference genome is either the genome of one individual or a consensus sequences from multiple individuals, it cannot include all variation within a population. Thus, when sequence reads are aligned to the reference genome, reads that match a reference genome allele tend to align with the reference genome at a higher frequency than reads comprising the allele variation that doesn't match the reference genome, regardless of the relative frequencies of the allele variant reads. Accordingly, there is a bias towards reads with alleles that are present in the reference genome. Therefore, at any given location, there is the change that an allele frequency (fraction of reads representing an allele) for the reference allele is more than that of the non-reference or alternate allele. In other words, there can be an underestimation of the frequencies of sequencing reads with a non-reference allele.

Some studies have shown that alignment bias occurs throughout the human genome, including at clinically-relevant locations. Thus, alignment bias can have significant implications for both researchers and clinicians.

One approach to address alignment bias is to align the reads to a plurality of reference genomes which comprise some of the variation within a population. However, not only is this approach slow and computationally expensive, but it cannot account for all variation among the many polymorphic locations within a population, and does not utilize the known variation of the genome being sequenced.

### SUMMARY OF THE DISCLOSURE

There is a continued need for methods and systems that reduce alignment bias resulting from next-generation sequencing.

The present disclosure is directed to inventive methods and systems for analyzing a genome. Various embodiments and implementations herein are directed to a system and method that aligns sequencing reads to a reference genome and uses the alignment to identify heterozygous locations, the variants at each heterozygous location, and the ratio of the variants at each heterozygous location. The system generates an alternate reference genome comprising the identified non-reference allele for each of the identified heterozygous locations, and aligns the same set of sequencing reads to the alternate reference genome. The ratio of variants at each of the identified heterozygous locations is then identified. The system then assesses and/or quantifies alignment bias at one or more of the identified heterozygous locations. According to an embodiment, the system assesses alignment bias by comparing the ratio of variants from the reference genome alignment to the ratio of variants from the alternate genome alignment.

Generally, in one aspect, is a method for analyzing a target genome using a genome analysis system. The method includes: (i) aligning sequencing data from the target genome to a reference genome; (ii) identifying, from the alignment, one or more heterozygous locations within the target genome, and wherein the identification further comprises an identification of allele variants and a frequency of each allele variants at each heterozygous location, the allele variants comprising both a reference allele variant also found in the reference genome and a non-reference allele variant not found in the reference genome; (iii) generating an alternate reference genome from the identification and from the reference genome, wherein the identified non-reference allele variant for one or more of the identified heterozygous locations replaces the reference allele variant in the reference genome; (iv) aligning sequencing data from the target genome to the alternate reference genome; (v) identifying, from the alignment, a frequency of the allele variants at each of the identified one or more heterozygous locations; (vi) assessing alignment bias at one or more of the identified heterozygous locations, comprising the step of comparing the frequency of allele variants from the reference genome alignment at an identified heterozygous location to the frequency of allele variants from the alternate genome alignment at that same location; and (vii) generating a report for a user comprising the assessment of alignment bias.

According to an embodiment, comparing comprises averaging the frequency of allele variants from the reference genome alignment with the frequency of allele variants from the alternate genome alignment to generate an averaged frequency for each allele variant. According to an embodiment, the report comprises: (i) each of the identified heterozygous locations; (ii) the frequency of allele variants from the reference genome alignment at an identified heterozygous location; (iii) the frequency of allele variants from the alternate genome alignment at that same location; and (iv) the averaged frequency for each allele variant.

According to an embodiment, the sequencing data from the target genome aligned to the reference genome is DNA sequencing data. According to an embodiment, the sequencing data from the target genome aligned to the alternate reference genome is the same DNA sequencing data aligned to the reference genome.

According to an embodiment, the sequencing data from the target genome aligned to the alternate reference genome is the RNA sequencing data.

According to an embodiment, the step of generating an alternate reference genome comprises generating a FASTA file.

According to an embodiment, a location is identified as heterozygous if the heterozygosity at that location meets or exceeds a pre-determined threshold. According to an embodiment, the pre-determined threshold for a variant allele location is based at least in part on a determined read depth at that location.

According to another aspect is a system configured to analyze a target genome. The system includes: a reference genome; a set of DNA sequencing data from a target genome; a processor configured to: (i) align sequencing data from the target genome to a reference genome; (ii) identify, from the alignment, one or more heterozygous locations within the target genome, wherein the identification further comprises an identification of allele variants and a frequency of each allele variants at each heterozygous location, the allele variants comprising both a reference allele variant also found in the reference genome and a non-reference allele variant not found in the reference genome; (iii) generate an alternate reference genome from the identification and from the reference genome, wherein the identified non-reference allele variant for one or more of the identified heterozygous locations replaces the reference allele variant in the reference genome; (iv) align sequencing data from the target genome to the alternate reference genome; (v) identify, from the alignment, a frequency of the allele variants at each of the identified one or more heterozygous locations; and (vi) assess alignment bias at one or more of the identified heterozygous locations, comprising the step of comparing the frequency of allele variants from the reference genome alignment at an identified heterozygous location to the frequency of allele variants from the alternate genome alignment at that same location; and a data structure configured to store the assessment of alignment bias.

According to an embodiment, the system further includes a set of RNA sequencing data from the target genome, and the processor is configured to align the RNA sequencing data to the alternate reference genome.

According to an embodiment, the processor is configured to assess alignment bias by averaging the frequency of allele variants from the reference genome alignment with the frequency of allele variants from the alternate genome alignment to generate an averaged frequency for each allele variant.

According to an embodiment, the system further includes a user interface configured to provide a report for a user comprising the assessment of alignment bias. According to an embodiment, the report comprises: (i) each of the identified heterozygous locations; (ii) the frequency of allele variants from the reference genome alignment at an identified heterozygous location; (iii) the frequency of allele variants from the alternate genome alignment at that same location; and (iv) an averaged frequency for each allele variant.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some o f the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for analyzing a genome, in accordance with an embodiment.

FIG. 2 is a schematic representation of a system for analyzing a genome, in accordance with an embodiment.

### DETAILED DESCRIPTION OF EMBODIMENTS

The present disclosure describes various embodiments of a system and method for analyzing genomic sequencing using an alternate reference genome generated from heterozygous alleles identified in the analyzed genome. More generally, Applicant has recognized and appreciated that it would be beneficial to provide a method that reduces alignment bias during sequencing read alignment. The system, which may optionally comprise a sequencing platform, generates or receives sequencing data comprising sequencing reads from a target genome. The reads are aligned to a reference genome to identify heterozygous locations, the variant alleles at each heterozygous location, and the ratio of the variant alleles at each heterozygous location. An alternate reference genome is generated using the identified non-reference allele variant for each of the identified heterozygous locations. The system then aligns the same set of reads to the generated alternate reference genome and identifies the ratio of variant alleles at the identified heterozygous locations. Alignment bias can be assessed or quantified by comparing the ratio of variants from the reference genome alignment to the ratio of variants from the alternate genome alignment for one or more of the identified heterozygous locations.

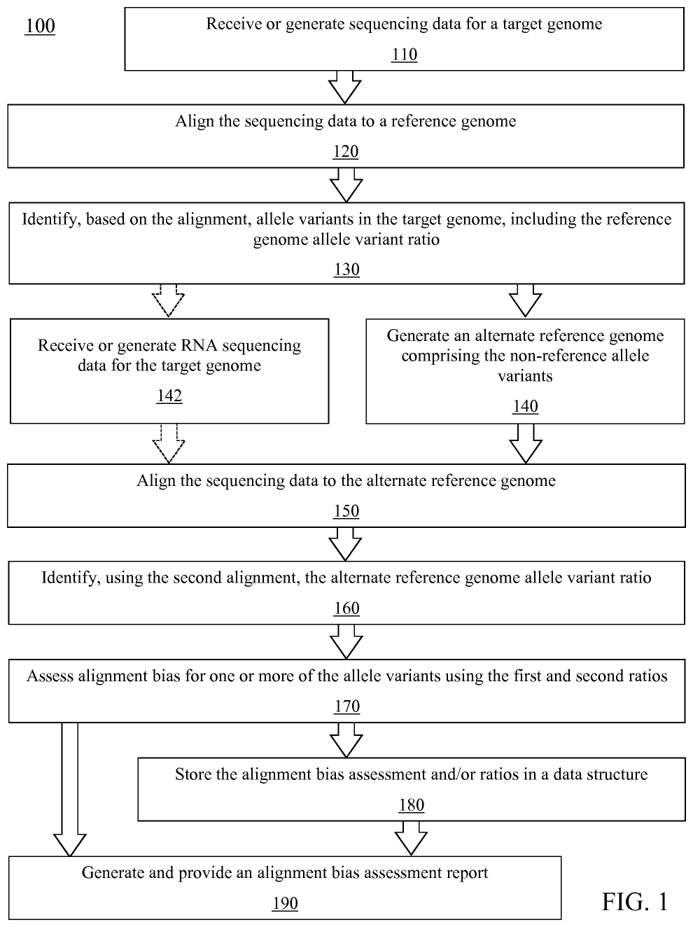

Referring to FIG. 1, in one embodiment, is a flowchart of a method 100 for reducing alignment bias using a genome analysis system. The genome analysis system may be any of the systems described or otherwise envisioned herein, and may comprise any of the components described or otherwise envisioned herein.

At step 110 of the method, the genome analysis system generates and/or receives sequencing data for a target genome. The target genome can be any genome from any organism, including pathogenic and non-pathogenic organisms. It is recognized that there is no limitation to the source of the target genome.

According to an embodiment, the genome analysis system comprises a sequencing platform configured to obtain sequencing data from the target genome. The sequencing platform can be any sequencing platform, including but not limited to any system described or otherwise envisioned herein. A sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner. According to an embodiment, the genome analysis system receives the sequencing data for the target genome. For example, the genome analysis system may be in communication or otherwise receive data from a database comprising one or more target genomes.

The generated and/or received sequencing data may be stored in a local or remote database for use by the genome analysis system. For example, the genome analysis system may comprise a database to store the sequencing data for the target genome, and/or may be in communication with a database storing the sequencing data. These databases may be located with or within the genome analysis system or may be located remote from the genome analysis system, such as in cloud storage and/or other remote storage.

The generated and/or received sequencing data may comprise a complete or mostly complete genome, or may be a partial genome. For example, the generated and/or received sequencing data may be assemblies, whole genome constructs, incomplete genomes, partial genomes, exomes, and/or any other sequencing data.

At step 120 of the method, the sequencing data is aligned with a reference genome. The reference genome used for the alignment may be any reference genome, such as a standard reference genome or a reference genome selected from a plurality of possible reference genomes. The reference genome may be obtained from a public or private database or storehouse of reference genomes, and may be in any format utilizable by the genome analysis system. According to an embodiment, the reference genome is a FASTA file, although many other file types are possible. Among other possibilities, the reference genome may be a graph-based genome.

The sequencing data, comprising a plurality of sequencing reads, is then aligned with the reference genome using any method of alignment, including but not limited to current and future alignment algorithms or methods. There are a variety of different tools available for sequence alignment, including both proprietary and open-source software, and any of these tools may be used to align the plurality of sequencing reads with the reference genome.

At step 130 of the method, the genome analysis system analyzes the sequence alignment to identify heterozygous locations. A heterozygous location may be any location along the reference genome where the alignment indicates that the target genome comprises multiple alleles, or variant alleles, at that location. Heterozygous locations may be identified using any variant calling algorithm, including but not limited to Varscan®, Samtools, and GATK®, among many others. For each heterozygous location, the variant calling algorithm may identify, for example, the location of the allele variant, the variant alleles at that location, and/or the frequencies of the variant alleles at that location. The variant alleles will typically comprise one allele corresponding to the reference genome (a “reference allele”) and a second, different allele (a “non-reference allele”).

According to an embodiment, the determination of heterozygosity at a location may be determined in part based on a pre-determined or variable threshold. Thus, a location may be determined to be heterozygous only if there is a high confidence variant identified at that location. The variant calling algorithm may, for example, require that a variant be identified in a minimum percentage of reads aligned at a location, where the minimum percentage can be or can be based on a pre-determined or variable threshold. The threshold may be programmed into the genome analysis system or may be determined or modified by a user of the genome analysis system, or by another system working with the genome analysis system.

According to an embodiment, for some applications the genome analysis system and/or variant calling algorithm may be programmed or otherwise instructed or designed to require that a variant be identified in at least 25% of reads at a location, such that variants identified in less than 25% of reads are considered noise and won't be identified as variant alleles ora heterozygous location. According to another application, such as one requiring a more stringent variant calling protocol, the genome analysis system and/or variant calling algorithm may be programmed or otherwise instructed or designed to require that a variant be identified in at least 40% of reads at a location.

According to an embodiment, the threshold may optionally be wholly or partially dependent upon the read depth at the analyzed location. For example, if there are 50 reads aligned across an analyzed location the minimum percentage may be 40%, while the minimum percentage may be 25% if there are 100 reads aligned across the analyzed location. Thus, the system may be programmed or otherwise instructed or designed to require at least a first percentage if there are fewer than a predetermined number of reads, and a second lower percentage if there are more than a predetermined number of reads. These and many other thresholds and variations may be programmed, selected, or otherwise determined by the system and/or by a user.

Referring to TABLE 1 is an example list of five variants identified by the genome analysis system. Each variant is associated with a location within the reference genome as well as the variants at that location found within the reads obtained from the target genome. For example, at position 20,650,507 on chromosome 1 the genome analysis system identified two alleles, an “A” (which in this example is the allele found within the selected reference genome) at approximately 53% and a “C” at approximately 47%. A variant or heterozygous location may optionally be associated with any other additional information.

Due to the possibility of alignment bias, it is unclear from this initial alignment whether the percentages represent the true frequencies of variant alleles at position 20,650,507 on chromosome 1, which would suggest a genotype of AC within the target genome, or whether, for example, the reference genome value of “A” might be noise and the genotype is more likely CC within the target genome.

TABLE 1
Variants identified by the genome analysis system

| | | Location | | | |
|---|---|---|---|---|---|
| ID | Chromosome | (GRCH38) | SNP ID | Variants | Frequency |
| 1 | 1 | 20,650,507 | rs1043424 | A (reference) | 53% |
| | | | | C | 47% |
| 2 | 1 | 109,499,822 | rs1043274 | C (reference) | 58% |
| | | | | T | 42% |
| 3 | 1 | 225,831,932 | rs1051740 | T (reference) | 65% |
| | | | | C | 35% |
| 4 | 2 | 105,281,283 | rs1020064 | G (reference) | 69% |
| | | | | T | 31% |
| 5 | 2 | 85,581,859 | rs1010 | A (reference) | 70% |
| | | | | G | 30% |

According to an embodiment, the genome analysis system generates an output from the analysis by the variant calling algorithm or method. The output may be, for example, any of the information generated by the variant calling algorithm or method. For example, the output may comprise one or more variant locations and the values of the variant alleles at each location. The output may comprise additional information, including but not limited to the frequencies of the variant alleles at each location, among other types of information. This output may be utilized in downstream functionality of the genome analysis system as described or otherwise envisioned herein.

At step 140 of the method, the genome analysis system generates an alternate reference genome from the identified variant alleles. According to an embodiment, the genome analysis system generates an alternate reference genome from high-confidence variant alleles, meaning heterozygous locations that have satisfied a minimum threshold for a variant allele.

According to an embodiment, the genome analysis system generates the alternate reference genome using the non-reference alleles at each of the identified heterozygous locations. For example, if the reference genome comprises allele “A” at position 20,650,507 on chromosome 1 and the genome analysis system identified a variant allele of “C” at this position, the genome analysis system generates the alternate reference genome with a C at position 20,650,507 on chromosome 1. According to an embodiment, the genome analysis system comprises an algorithm, such as a script, that utilizes variant locations and the values of the variant alleles at each location, as well as the FASTA file for the reference genome, to generate the alternate reference genome. For example, the script can modify the text-based FASTA file by substituting or replacing the reference allele with the variant allele at each identified heterozygous location, such as at each identified high-confidence heterozygous location. According to an embodiment, the genome analysis system generates the alternate reference genome using only non-reference alleles that are found above a certain percentage of reads. This minimum threshold can be defined by the genome reference system, by a user, or by other mechanisms.

According to an embodiment, the script or other algorithm generates an alternate reference genome and provides the genome as an output that is stored in temporary and/or long-term memory for downstream functionality of the genome analysis system. For example, the output ofthe script or other algorithm is a FASTA file, although many other file types are possible. Thus, the output of a script may be a FASTA file that is different from the reference genome FASTA file in that it comprises a variant allele at one or more of the identified heterozygous locations.

At step 150 of the method, the genome analysis system aligns sequencing data with the alternate reference genome. The genome analysis system can align the sequencing data with the alternate reference genome using any method of alignment, including but not limited to current and future alignment algorithms or methods. There are a variety of different tools available for sequence alignment, including both proprietary and open-source software, and any of these tools may be used to align the sequencing data with the reference genome.

The sequencing data aligned with the alternate reference genome may be the same DNA sequencing data aligned with the original reference genome. Alternatively, the sequencing data may be different DNA sequencing data. As yet another option, the sequencing data may be RNA sequencing data obtained from a wide variety of methods for obtaining sequencing data that can be aligned with the alternate reference genome.

Accordingly, at optional step 142 of the method, which may be performed together with or separate from step 110 of the method, the genome analysis system received or generates RNA sequencing data for the target genome. According to an embodiment, the genome analysis system comprises a sequencing platform configured to obtain the RNA sequencing data from the target genome. The RNA sequencing platform can be any RNA sequencing platform, including but not limited to any system described or otherwise envisioned herein. The generated and/or received RNA sequencing data may be stored in a local or remote database for use by the genome analysis system. For example, the genome analysis system may comprise a database to store the RNA sequencing data for the target genome, and/or may be in communication with a database storing the RNA sequencing data. These databases may be located with or within the genome analysis system or may be located remote from the genome analysis system, such as in cloud storage and/or other remote storage.

At step 160 of the method, the genome analysis system analyzes the sequence alignment to identify the allele frequencies at one or more of the identified heterozygous locations used to generate the alternate reference allele. The allele frequencies may be identified using any variant calling algorithm, including but not limited to Varscan®, Samtools, and GATK®, among many others. For each heterozygous location, the variant calling algorithm may identify, for example, the location of the allele variant, the variant alleles at that location, and/or the frequencies of the variant alleles at that location.

As with the first alignment, the variant calling algorithm may be programmed or otherwise modified or set to only identify and/or report variant allele frequencies at a heterozygous location if the minor frequency exceeds a minimum threshold. The variant calling algorithm may, for example, require that a variant be identified in a minimum percentage of reads aligned at a location, where the minimum percentage can be or can be based on a pre-determined or variable threshold. The threshold may be programmed into the genome analysis system or may be determined or modified by a user of the genome analysis system, or by another system working with the genome analysis system.

Referring to TABLE 2 is an example list of a variant identified by the genome analysis system in the alternate reference genome, which was also identified in the alignment of the original reference genome. The variant is associated with a location within the alternate reference genome as well as the variant alleles found at that location found within the reads obtained from the target genome. Thus, at position 20,650,507 on chromosome 1 the genome analysis system identified two alleles, the “C” allele (which in this example is the allele found within the alternate reference genome) at approximately 75% and the “A” allele at approximately 47%. A variant or heterozygous location may optionally be associated with any other additional information.

TABLE 2
Variants identified by the genome analysis system

| | | Location | | | |
|---|---|---|---|---|---|
| ID | Chromosome | (GRCH38) | SNP ID | Variants | Frequency |
| 1 | 1 | 20,650,507 | rs1043424 | C (alternate | 75% |
| | | | | reference) | |
| | | | | A | 25% |

According to an embodiment, the genome analysis system generates an output from the analysis by the variant calling algorithm or method. The output may be, for example, any of the information generated by the variant calling algorithm or method. For example, the output may comprise variant allele values and frequencies at one or more of the identified heterozygous locations. This output may be utilized in downstream functionality of the genome analysis system as described or otherwise envisioned herein.

At step 170 of the method, the genome analysis system assesses and/or quantifies alignment bias at one or more identified heterozygous locations. According to an embodiment, the genome analysis system assesses and/or quantifies alignment bias by comparing: (1) the variant allele frequencies obtained during alignment of sequencing data from the target genome with the original reference genome, and (2) the variant allele frequencies obtained during alignment of sequencing data from the target genome with the alternate reference genome. Referring to TABLE 3, in one embodiment, is a comparison of the allele variant frequencies obtained from the two alignments.

TABLE 3
Comparison of allele frequencies

| | | | | | Fre- | Fre- |
|---|---|---|---|---|---|---|
| | | | | | quency | quency |
| | | | | | (Refer- | (Alter- |
| | Chromo- | Location | | | ence | nate |
| ID | some | (GRCH38) | SNP ID | Variants | Genome) | Genome) |
| 1 | 1 | 20,650,507 | rs1043424 | A | 53% | 25% |
| | | | | (refer- | | |
| | | | | ence) | | |
| | | | | C | 47% | 75% |
| | | | | (alternate | | |
| | | | | reference) | | |

According to an embodiment, the assessment and/or quantification of alignment bias at a heterozygous location comprises averaging the allele variant frequencies from the two alignments. For example, referring to TABLE 4, in one embodiment, is an example of averaging of the allele variant frequencies from the first and second alignments. The average frequency for the reference “A” allele variant at position 20,650,507 on chromosome 1 is 39%, while the average frequency for the “C” allele variant at position 20,650,507 on chromosome 1 is 61%. This information may be utilized by the genome analysis system, by a user, and/or by any other methodology or system.

TABLE 4
Analysis of allele frequencies

| | | | | | Fre- | Fre- | Aver- |
|---|---|---|---|---|---|---|---|
| | | | | | quency | quency | age |
| | Chro- | | | | (Refer- | (Alter- | Fre- |
| | mo- | Location | | Vari- | ence | nate | quen- |
| ID | some | (GRCH38) | SNP ID | ants | Genome) | Genome) | cy |
| 1 | 1 | 20,650,507 | rs1043424 | A | 53% | 25% | 39% |
| | | | | (refer- | | | |
| | | | | ence) | | | |
| | | | | C | 47% | 75% | 61% |
| | | | | (alter- | | | |
| | | | | nate | | | |
| | | | | refer- | | | |
| | | | | ence) | | | |

According to an embodiment, the genome analysis system may identify alleles and/or locations that, based on the assessment and/or quantification of alignment bias, show no indication of alignment bias. In other words, the system may identify one or more alleles and/or locations, such as all alleles and/or locations, where the frequencies for the alignment with the reference genome and the frequencies for the alignment with the alternate genome were identical, nearly identical, or having similarity lower than a maximum threshold. For example, the system may identify alleles and/or locations where the difference between the two frequencies is zero, almost zero, or below a threshold such as 5%, 10%, 25%, or another possible threshold. The threshold can be determined, for example, by the genome analysis system, by a user, and/or by another system.

According to an embodiment, the genome analysis system generates an output from the assessment and/or quantification of alignment bias at one or more of the heterozygous location. For example, the system may generate an output comprising an allele frequency for the reference genome at each heterozygous location, an allele frequency for the alternate genome at each heterozygous location, and/or the averaged allele frequency at each heterozygous location. More or less information may be provided in the output depending on the programming, settings, or other design of the genome analysis system. According to another example, the system may generate an output comprising the identification of alleles and/or locations that, based on the assessment and/or quantification of alignment bias, show no or little indication of alignment bias as described or otherwise envisioned herein.

At step 180 of the method, the genome analysis system may generate, from the assessment and/or quantification of alignment bias, a report of allele frequency obtained by the method described or otherwise envisioned herein. For example, the report may comprise a text-based file or other format comprising information such as the allele frequency for the reference genome at each heterozygous location, the allele frequency for the alternate genome at each heterozygous location, and/or the averaged allele frequency at each heterozygous location, although any other information obtained by or from the genome analysis system, the sequencing data, the target or reference genomes, and/or other sources may be included in the report.

For example, the genome analysis system may visually display information about one or more of the heterozygous locations on a screen or other display method. A clinician or researcher may only be interested in one or several heterozygous locations, and thus the genome analysis system may be instructed or otherwise designed or programmed to only display information obtained for the one or several heterozygous locations.

According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a researcher, clinician, or other user to review and implement an action or response based on the provided information. For example, a researcher or clinician may utilize the information to mine for variant alleles in the target genome, such as a genome of a patient or a research subject. The user may manually review the report to identify all variant alleles, or to identify specific variant alleles, or may use software or other methodology to identify one or more variant alleles. Identifying variant alleles is an important aspect of disease research, disease diagnosis, and disease treatment. Accordingly a clinician may, for example, diagnose a genetic disorder or hypothesize the existence of a particular genetic disorder based on the output of the report. The clinician may additional or alternatively select a specific treatment based on the output of the report.

As another example, a user may review the report or information to determine whether specific locations within the target genome comprise variant alleles. For example, a researcher, clinician, or other user may be interested in specific variant alleles for research, treatment, or other purposes and may review a report and/or generate a report directed to the allele locations of interest. The existence or absence of an allele variant, as indicated by the report, provides the necessary research or treatment information for the user. Many other downstream uses are possible.

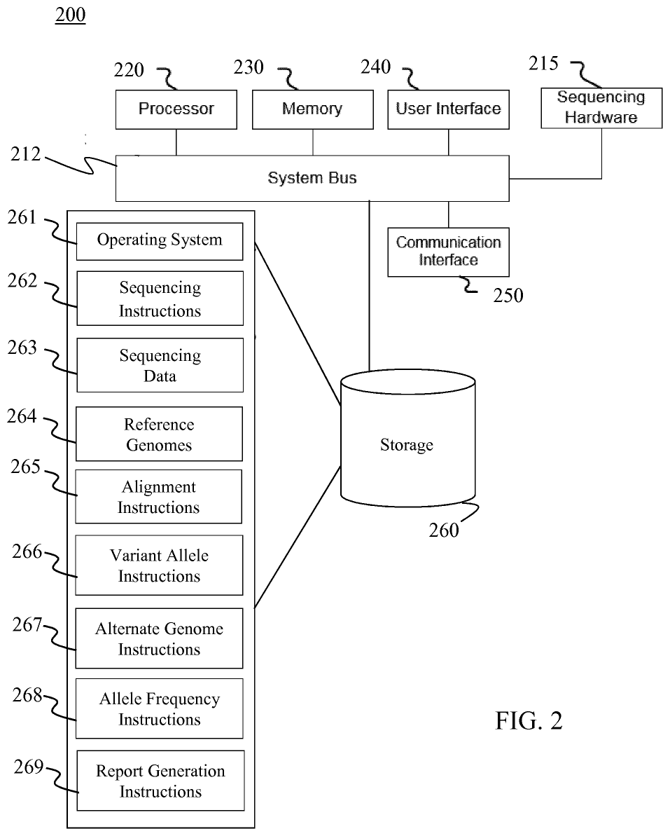

Referring to FIG. 2, in one embodiment, is a schematic representation of a genome analysis system 200 configured to analyze alignment bias. System 200 may be any of the systems described or otherwise envisioned herein, and may comprise any of the components described or otherwise envisioned herein.

According to an embodiment, system 200 comprises one or more of a processor 220, memory 230, user interface 240, communications interface 250, and storage 260, interconnected via one or more system buses 212. In some embodiments, such as those where the system comprises or directly implements a sequencer or sequencing platform, the hardware may include additional sequencing hardware 215. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 360 may store instructions for execution by processor 220 or data upon which processor 220 may operate. For example, storage 260 may store an operating system 261 for controlling various operations of system 200. Where system 200 implements a sequencer and includes sequencing hardware 215, storage 260 may include sequencing instructions 262 for operating the sequencing hardware 215, and sequencing data 263 obtained by the sequencing hardware 215, although sequencing data 263 may be obtained from a source other than an associated sequencing platform.

Storage 260 may also store one or more reference genomes 264, and/or system 200 may be in communication with a reference genome database. A reference genome database may be a public database or a private database and may be stored remotely and accessed via the communication interface. The reference genome database may comprise one or more reference genomes.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While genome analysis system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of genome analysis system 200 may store one or more algorithms and/or instructions to carry out one or more functions or steps of the methods described or otherwise envisioned herein. For example, processor 220 may comprise alignment instructions or software 265, variant allele calling instructions or software 266, alternate reference genome instructions or software 267, allele frequency instructions or software 268, and/or report generation instructions or software 269, among many other algorithms and/or instructions to carry out one or more functions or steps of the methods described or otherwise envisioned herein.

According to an embodiment, alignment instructions or software 265 direct the system to align the sequence data with a reference genome. The sequence data may be any sequence data from a target genome, and may be generated or otherwise obtained by the system. For example, the genome analysis system may comprise a sequencing platform configured to obtain sequencing data from the target genome, or may be in communication with or otherwise receive sequencing data generated by another system from the target genome. The generated and/or received sequencing data may be stored in a local or remote database for use by the genome analysis system. The generated and/or received sequencing data may comprise a complete or mostly complete genome, or may be a partial genome. For example, the generated and/or received sequencing data may be assemblies, whole genome constructs, incomplete genomes, partial genomes, exomes, and/or any other sequencing data.

The reference genome used by the system for the alignment may be any reference genome, such as a standard reference genome or a reference genome selected from a plurality of possible reference genomes. The reference genome may be stored by the system or may be obtained, retrieved, or otherwise received by the system. According to an embodiment the reference genome is a FASTA file, although many other file types are possible.

Once the system has the sequencing data and a reference genome, the alignment instructions or software 265 direct the system to align the sequencing data with a reference genome. The sequencing data is aligned with the reference genome using any method of alignment, including but not limited to current and future alignment algorithms or methods. There are a variety of different tools available for sequence alignment, including both proprietary and open-source software, and any of these tools may be used to align the plurality of sequencing reads with the reference genome. Accordingly, system 200 may comprise proprietary and/or open-source software or algorithms configured to align the sequencing data with the reference genome. The alignment instructions or software 265 therefore instruct system 200 to generate a genome alignment utilized by other functionality of the system.

As described herein, the alignment instructions or software 265 may also direct the system to align the same or other sequencing data with an alternate genome. Thus, the alignment instructions may direct system 200 to align the DNA sequencing data with an alternate genome generated by the system, or may direct the system to align RNA sequencing data with the alternate genome, and/or may direct the system to align any other sequencing data with the alternate genome.

According to an embodiment, variant allele calling instructions or software 266 direct the system to identify variants in the genome alignment. Variants may be identified using any variant calling method, including but not limited to Varscan, Samtools, and GATK, among many others. The variant allele calling instructions or software 266 may therefore comprise proprietary and/or open-source software or algorithms. The instructions may direct the system to identify, for example, the location of an allele variant, the variant alleles at that location, and/or the frequencies of the variant alleles at that location. The variant alleles will typically comprise one allele corresponding to the reference genome and a second, different allele.

According to an embodiment, variant allele calling instructions or software 266 direct the system to only identify variants that satisfy a certain threshold, thus being high-confidence variants. The variant calling algorithm may, for example, require that a variant be identified at a minimum frequency such as 25%, 50%, 75%, or any other percentage. This may be dependent upon the read depth of the variant location as described herein. The threshold may be programmed, selected, or otherwise determined by the system and/or by a user. For example, a user may select a frequency threshold via user interface 240, among other input methods.

According to an embodiment, alternate reference genome instructions or software 267 direct the system to generate an alternate reference genome from the identified variant alleles. According to an embodiment, the genome analysis system generates an alternate reference genome from high-confidence variant alleles, meaning heterozygous locations that have satisfied a minimum frequency threshold for the allele variant. The alternate reference genome instructions or software 267 may be, for example, an algorithm such as a script that utilizes variant locations and the values of the variant alleles at each location, as well as a file for the reference genome, to generate the alternate reference genome. For example, the script can modify a text-based FASTA file by substituting or replacing the reference allele with the variant allele at each identified heterozygous location, such as at each identified high-confidence heterozygous location.

According to an embodiment, the alternate reference genome instructions or software 267 direct the system to generate the alternate reference genome and provide the genome as an output that is stored in temporary and/or long-term memory for other functionality of the genome analysis system. For example, the output of the instructions may be a FASTA file, although many other file types are possible.

According to an embodiment, allele frequency instructions or software 268 direct the system to assess and/or quantify alignment bias at one or more identified heterozygous locations. According to just one embodiment, the genome analysis system assesses and/or quantifies alignment bias by comparing: (1) the variant allele frequencies obtained during alignment of sequencing data from the target genome with the original reference genome, with (2) the variant allele frequencies obtained during alignment of sequencing data from the target genome with the alternate reference genome. Many other types of assessments and quantifications are possible. According to an embodiment, the allele frequency instructions or software 268 may direct the system to average the allele variant frequencies from the two alignments.

As another example, the allele frequency instructions or software 268 may direct the system to identify alleles and/or locations that, based on the assessment and/or quantification of alignment bias, show no indication of alignment bias. For example, the system may identify alleles and/or locations where the difference between the two frequencies is zero, almost zero, or below a threshold such as 5%, 10%, 25%, or another possible threshold. The threshold can be determined, for example, by the genome analysis system, by a user, and/or by another system.

The allele frequency instructions or software 268 may direct the system to generate an output from the assessment and/or quantification of alignment bias. For example, the system may generate an output comprising an allele frequency for the reference genome at each heterozygous location, an allele frequency for the alternate genome at each heterozygous location, and/or the averaged allele frequency at each heterozygous location. According to another example, the system may generate an output comprising the identification of alleles and/or locations that, based on the assessment and/or quantification of alignment bias, show no or little indication of alignment bias as described or otherwise envisioned herein.

According to an embodiment, report generation instructions or software 269 direct the system to generate a user report comprising information about the analysis performed by the system. For example, a report may comprise information about the assessment and/or quantification of alignment bias, such as a report of allele frequency or frequencies.

The report may be generated for any format or output method, such as a file format, a visual display, or any other format. A report may comprise a text-based file or other format comprising information such as the allele frequency for the reference genome at each heterozygous location, the allele frequency for the alternate genome at each heterozygous location, and/or the averaged allele frequency at each heterozygous location, although any other information obtained by or from the genome analysis system, the sequencing data, the target or reference genomes, and/or other sources may be included in the report.

The report generation instructions or software 269 may direct the system to store the generated report or information in temporary and/or long-term memory or other storage. This may be local storage within system 200 or associated with system 200, or may be remote storage which received the report or information from or via system 200. Additionally and/or alternatively, the report or information may be communicated or otherwise transmitted to another system, recipient, process, device, and/or other local or remote location.

The report generation instructions or software 269 may direct the system to provide the generated report to a user or other system. For example, the genome analysis system may visually display information about one or more of the heterozygous locations on the user interface, which may be a screen or other display. A clinician or researcher may only be interested in one or several heterozygous locations, and thus the genome analysis system may be instructed or otherwise designed or programmed to only display information obtained for the one or several heterozygous locations.

The genome analysis system and approach described or otherwise envisioned herein provides numerous advantages over existing systems and methods. For example, current systems and methods fail to address the alignment bias that occurs throughout the human genome, including at clinically-relevant locations. This alignment bias can have significant implications for both researchers and clinicians, as it can obscure the actual genotype of the target genome. The methods and systems described or otherwise envisioned herein reduce alignment bias through the use of an alternate genome alignment.

According to an embodiment, the genome analysis system and approach described or otherwise envisioned herein enables a researcher, clinician, or other user to more accurately determine the genotype of the target genome, and thus to implement that information in research, diagnosis, treatment, and/or other decisions. This significantly improves the research, diagnosis, and/or treatment decisions of the researcher, clinician, or other user.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for analyzing a target genome using a genome analysis system, comprising:
aligning sequencing data from the target genome to a reference genome;
from the aligned sequencing data identifying, one or more heterozygous locations within the target genome the heterozygous locations comprising any location along the reference genome where the aligned sequencing data indicates that the target genome comprises multiple alleles or variant alleles at that location. and identifying allele variants and a frequency of each allele variants at each heterozygous location, the allele variants comprising both a reference allele variant also found in the reference genome and a non-reference allele variant not found in the reference genome;
generating an alternate reference genome from the identification of heterozygous locations, identification of allele variants and from the reference genome, wherein the identified non-reference allele variant for one or more of the identified heterozygous locations replaces the reference allele variant in the reference genome;
aligning sequencing data from the target genome to the alternate reference genome;
identifying, from the alignment to the alternate reference genome, a frequency of the allele variants at each of the identified one or more heterozygous locations;
assessing alignment bias at one or more of the identified heterozygous locations, comprising the step of comparing the frequency of allele variants from the reference genome alignment at an identified heterozygous location to the frequency of allele variants from the alternate genome alignment at that same location; and
generating a report for a user comprising the assessment of alignment bias.

2. The method of claim 1, wherein comparing comprises averaging the frequency of allele variants from the reference genome alignment with the frequency of allele variants from the alternate genome alignment to generate an averaged frequency for each allele variant.

3. The method of claim 2, wherein the report comprises: (i) each of the identified heterozygous locations; (ii) the frequency of allele variants from the reference genome alignment at an identified heterozygous location; (iii) the frequency of allele variants from the alternate genome alignment at that same location; and (iv) the averaged frequency for each allele variant.

4. The method of claim 1, wherein the sequencing data from the target genome aligned to the reference genome is DNA sequencing data.

5. The method of claim 4, wherein the sequencing data from the target genome aligned to the alternate reference genome is the same DNA sequencing data aligned to the reference genome.

6. The method of claim 1, wherein the sequencing data from the target genome aligned to the alternate reference genome is the RNA sequencing data.

7. The method of claim 1, wherein the step of generating an alternate reference genome comprises generating a FASTA file.

8. The method of claim 1, wherein a location is identified as heterozygous if the heterozygosity at that location meets or exceeds a pre-determined threshold.

9. The method of claim 8, wherein the pre-determined threshold for a variant allele location is based at least in part on a determined read depth at that location.

10. A system (o configured to analyze a target genome, comprising:
a reference genome;
a set of DNA sequencing data from a target genome;
a processor configured to: (i) align sequencing data from the target genome to a reference genome; (ii) identify, from the aligned sequencing data, one or more heterozygous locations within the target genome, the heterozygous locations comprising any location along the reference genome where the aligned sequencing data indicates that the target genome comprises multiple alleles or variant alleles at that location, the processor also being configured to identify allele variants and a frequency of each allele variants at each heterozygous location, the allele variants comprising both a reference allele variant also found in the reference genome and a non-reference allele variant not found in the reference genome; (iii) generate an alternate reference genome from the identification of heterozygous locations, identification of allele variants and from the reference genome, wherein the identified non-reference allele variant for one or more of the identified heterozygous locations replaces the reference allele variant in the reference genome; (iv) align sequencing data from the target genome to the alternate reference genome; (v) identify, from the alignment to the alternate reference genome, a frequency of the allele variants at each of the identified one or more heterozygous locations; and (vi) assess alignment bias at one or more of the identified heterozygous locations, comprising the step of comparing the frequency of allele variants from the reference genome alignment at an identified heterozygous location to the frequency of allele variants from the alternate genome alignment at that same location; and
a data structure configured to store the assessment of alignment bias.

11. The system of claim 10, further comprising a set of RNA sequencing data from the target genome, wherein the processor is configured to align the RNA sequencing data to the alternate reference genome.

12. The system of claim 10, wherein the processor is configured to assess alignment bias by averaging the frequency of allele variants from the reference genome alignment with the frequency of allele variants from the alternate genome alignment to generate an averaged frequency for each allele variant.

13. The system of claim 10, wherein the processor is configured to identify a location as heterozygous if the heterozygosity at that location meets or exceeds a pre-determined threshold.

14. The system of claim 10, further comprising a user interface configured to provide a report for a user comprising the assessment of alignment bias.

15. The system of claim 14, wherein the report comprises: (i) each of the identified heterozygous locations; (ii) the frequency of allele variants from the reference genome alignment at an identified heterozygous location; (iii) the frequency of allele variants from the alternate genome alignment at that same location; and (iv) an averaged frequency for each allele variant.
